(19) United States
(12) Patent Application Publication
(10) Pub. No.: US 2015/0031006 A1
(43) Pub. Date: Jan. 29, 2015

(54) METHOD FOR ALTERING RECURRING DREAMS INCLUDING NIGHTMARES

(71) Applicant: Timothy Charles Melkus, Santa Monica, CA (US)

(72) Inventor: Timothy Charles Melkus, Santa Monica, CA (US)

(21) Appl. No.: 14/458,865

(22) Filed: Aug. 13, 2014

Related U.S. Application Data
(63) Continuation-in-part of application No. 13/852,056, filed on Mar. 28, 2013, now abandoned.

(51) Int. Cl. A61M 21/02; G09B 19/00

(52) U.S. Cl. CPC A61M 21/02; G09B 19/00; A61M 2021/0011
USPC 434/236

(57) ABSTRACT

The invention provides a method of altering dreams, including nightmares, which can be applied to individuals or groups.

### PRIORITY

This application is a continuation-in-part of U.S. patent application Ser. No. 13/852,056, which is incorporated in its entirety herein my reference.

### FIELD OF THE INVENTION

The present invention relates to a method of dream, including nightmare, management and more particularly, the present invention is directed to the alteration of recurring dreams, including nightmares.

### BACKGROUND OF THE INVENTION

Since the beginning of human awareness, dreams have likely existed “as a short movie played out in the theater of the mind; . . . [t]he film is often not linear but circular, existing without time and space.” U.S. patent application Ser. No. 11/380,069, Publication No. 20060241336 (published Oct. 26, 2006) (Jonathan D. Gershon, applicant). To Sigmund Freud, “the dream thinks in pictures.” Sigmund Freud, The Interpretation of Dreams. (3^{rd} ed. 1915). Before movies and pictures, there were books, plays, paintings, drawings, hieroglyphs and etchings. Depictions exist on everything from cave walls dating back far before recorded history to digits in ether today. What are all these depictions regardless of technology, form or time? Like many movies and pictures, early cave drawings largely detail scenes of ordinary people and animals. Individuals, groups and tribes are portrayed. Domesticated and game animals of all forms and proportions are portrayed. Some of these animals still exist, while others disappeared long ago. Mostly, these drawings appear to be familiar recollections of daily life. Yet there is ample evidence that from the very beginning, like today, every so often the tales told in the theaters of prehistoric minds and scratched or painted onto cave walls crossed the line from images of daily life to tales of terrible trauma, fear and reverence. Common yet surreal themes depicted all through the ages include scenes of hunts and battles or gods and aliens. These depictions are powerful primordial images representing feelings that have spawned the demons that have come in the night to men and women since time began. In those early days, on prairies and in caves, our ancestors could only pray to the Gods or silently suffer as their nightmares haunted them on a whim. Wikipedia, the Free Encyclopedia, Antti Revonsuo at http://en.wikipedia.org/w/index.php?title=Antti_Revonsuo&oldid=513810639. As the ages have slowly passed we are now becoming aware that we can use our dreams and nightmares productively, in a systematic fashion, to set us free and empower us to learn, adapt and evolve.

Dreams have always both guided and limited everyday men and women as well as conquerors and kings Throughout most of human history, dream therapy was fundamentally more art than science; practiced by holy men and healers and often involving aids such as baths, vapors and snakes. More recently, written works on dream therapy were developed including the dream diagnoses contained in the 19^{th} dynasty Egyptian book, “The Dream Book”, from around 1275 B.C and the dictionaries and descriptions of Artemidorus Daldianus in the second century A.D. The British Museum, The Dream Book at http://www.britishmuseum.org/explore/highlights/highlight_objects/aes/t/the_dream_book.aspx; Wikipedia, the Free Encyclopedia, Artemidorus at http://en.wikipedia.org/wiki/Artemidorus. However, the seminal work wrestling dream interpretation away from the heavenly arts and subjecting it to earthly human order occurred only 114 years ago with Dr. Sigmund Freud's “The Interpretation of Dreams.” Freud, supra. Since his seminal work, the world has embraced, discarded, and altered Freud's dream perspectives. Despite the array of activity, overall efforts are unsystematic and true innovation has been slow. This disjointed approach has caused dream and nightmare terminology, interpretation and resolution to become even more splintered than before with a conflicting variety of available practitioners, books, blogs, web sites, forums, etc. See, e.g., Dream Yoga at http://dream-yoga.org/dream-interpretation/useless-dreams; http://www.amiracleaday.com/articles/2007/08/09/how-to-get-rid-of-bad-dreams-in-one-simple-step; and http://www.wikihow.com/Get-Rid-of-Bad-Dreams.

All dream and nightmare therapy efforts available today may conceptually be placed under the umbrella of the disparate arts or Freudian therapies. With the welcome addition of modern psychology-based understandings to this fundamentally unstructured domain, however, a Gordian Knot of art and science now exists. Dream terminologies, for example, now range from the fine works of Artemidorus to Freud's balletic flow of psychic energy among conscious, unconscious and preconscious (later id, ego and superego, respectively). This mix includes both congruent terms and interpretations for a given dream as well as terms and interpretations diametrically opposed to one another and everything in between.

There is still no vision for how dream alteration, management, therapy and resolution can stay true to Hippocrates's Oath to “never do harm” and contribute to a kinder, gentler world for human evolution. Due partially to the branch of medicine in which dream therapy was developed and partially to the consensus that, regarding dreams, everyone truly is unique and special after all, there is no national or international database of scientifically generated, useable, and verifiable dream data. Compounding the problem is the lack of recognized dream disorders and treatments. The International Classification of Diseases, for example, contains over 69,000 diagnosis and procedure codes and only four apply to dreams: 1) F514—Sleep terrors, 2) F515—Nightmare disorder, 3) F518—Other sleep disorders not due to a substance or known physiological condition, and 4) F519—Sleep disorder not due to a substance or known physiological condition, unspecified. World Health Organization, (2013). ICD-10: International statistical classification of diseases and related health problems (10th Rev. ed.). New York, N.Y. The current Diagnostic and Statistical Manual of Mental Disorders contains three dream related disorders, allowing only a glimpse at prevalence and course of therapy in certain age groups: 1) 307.47—Nightmare Disorder (formerly Dream Anxiety Disorder), 2) 307.46—Sleep Terror Disorder, and 3) 307.47—Parasomnia, Not Otherwise Specified. American Psychiatric Association, (2000). Diagnostic and Statistical Manual of Mental Disorders (4th ed., text rev.). Washington, D.C. Treatment paradigms essentially do not exist for dreams, thus, baffling even the most fundamental analytical exploration pursuing insights. See, e.g., http://www.cms.gov/icd10manual/fullcode_cms/P1247.html and See, e.g., http://dsm.psychiatryonline.org/content.aspx?bookid=22&sectionid=1892296#10732. The fundamental brilliance of the gift of dreams is that each unique DNA that has ever existed absorbs and stores everything that it has ever been exposed to in its life and radiates all that uniqueness relatively freely each night; making each person's dreams truly their own. Since each person's dreams are unaware of the impossible, dictionaries and diagnoses should still be applied individually, and it is understandable that the subject of dreams may be one of the latter frontiers humans will ever fully map. Concurrently, patterns do exist across dreams and dreamers and these patterns allow dream management.

When Freud's cause and effect construct is distilled to, (1) feelings create dreams and (2) to get rid of dreams, get rid of or resolve the unresolved feelings that create (d) the dream, it becomes apparent that Freudian therapy offers no other way to alter dreams or reduce the recurrence and severity of unwanted dreams and nightmares. This constraint leaves the afflicted with long-term hope, but in the near-term they are largely unsatisfied and underserved. Freudian therapy also seems to tolerate no possibility to use dreams personally or professionally. Legend has it that Alexander the Great fashioned the “Alexandrian solution” by unleashing the product of the science and technology of his time, his mighty sword, to slice through an intractable problem. The present invention uses the science and technology of today to untie and demystify the Gordian Knot of dream management by focusing on manipulating the mechanism of dreams directly as they play in the unconscious mind instead of focusing solely on resolving the feelings that create dreams, thus providing a much-needed additional dream treatment tool.

Have you ever had characters from one dream appear in a completely separate dream? Most people have. Unconscious thoughts, including dreams playing as movies in the dreamer's mind each night, can intersect or bump into each other for a moment or an entire dream. Perhaps one dream includes a couple once met on a tropical island holiday while an entirely separate dream involves a business meeting in the near future. That island trip was wonderful; exotic, warm and soothing and they were cool and fun folks while the business meeting is an important upcoming pitch. More than one person has had the dream that while in the business meeting and looking around that island couple in Aloha shirts is sitting at the boardroom table smiling and waving. They may stay for the duration of this dream or they may appear in a flash. All that happened is that the two thoughts that were essentially separate and unrelated in the dreamer's memory and conscious mind became separate thoughts or dreams circulating in the unconscious mind, and the unconscious thoughts and dream fragments bumped into each other and intertwined.

A small subset of dream therapy or education relied on utilizing the dream state to affect the conscious state, attempting to splice unconscious thoughts with dreams. One such therapy was nocturnal learning tapes (i.e. intoning foreign languages, self-help messages, etc.). Nocturnal learning tapes used recordings of another speaker to imbue information to the user during sleep but proved untenable.

An even smaller subset focuses on the dream itself. One such therapy termed Imagery Rehearsal Therapy (“IRT”) uses a method of changing the entire ending of nightmares by rehearsing a script. IRT has been used to treat a range of dream issues from children's nightmares to post-traumatic stress disorder (“PTSD”). Dawn Huebner, Ph.D. What to Do When You Dread Your Bed, A Kid's Guide to Overcoming Problems With Sleep (2008). R. Nisha Aurora et al., (2010) Best Practice Guide for the Treatment of Nightmare Disorder in Adults, J Clin. Sleep Med. 6(4), 395. It should be noted that there is potential for IRT to intensify PTSD. Id. Another instance of focusing on the dream itself is a 2008 patent application that is directed to an apparatus that uses audible signaling during sleep (the dreamer's recorded voice asking questions in hopes the question connects with the dream.) U.S. patent application Ser. No. 12/148,865, Publication No. 20080269545 (published Oct. 30, 2008) (Brent E. Logan, applicant).

Limitations related to the current state of the art include: (1) successfully imbedding and maintaining long scripted paths that are necessary to successfully apply IRT and (2) the audible-to-conscious-to-unconscious signaling path and precise timing (requiring rapid eye movement detection (“REM”) capability attempting to synchronize message delivery either before or while the target dream is actually playing) needed for recorded voice technology; and (3) the lack of ability to introduce negative thoughts because they are blocked by the dreamer's indigenous defenses (likely due to the translation chain needed from audible-to-conscious-to-unconscious).

There is a need in the art for an effective method of dream management and therapy that uses easily imbedded and maintained phrases and does not include the need for machines or REM detection. Ideally, this method would not be limited in the tone or synchronization of the message to be introduced because the message would incorporate and bypass the dreamer's indigenous defenses. Imbedded

### SUMMARY OF THE INVENTION

In one embodiment, the invention relates to methods for altering a recurring dream comprising the steps of identifying an individual that has a recurring dream in need of alteration; gathering data from the individual, wherein the data comprises a map of the dream in a timeline form; inputting the gathered data into a Dream Alteration System (“DAS”) plan, wherein the DAS plan is presented in a form selected from the group consisting of a computer, book, CD, patent, patent application publication, and DVD; identifying a key decision point in the recurring dream by eliciting a response from the individual and referencing the DAS for suggestions; identifying alternative paths from the key decision point by eliciting a response from the individual; choosing a specific path from among the alternative paths; determining a phrase to initiate the specific path by referencing the DAS plan for suggestions and eliciting a response from the individual; inputting the selected path and phrase into the DAS plan; creating an actionable dream strategy for modifying the outcome of the recurring dream, wherein the strategy includes the individual placing a physical copy of the DAS plan, or a representation of the plan on a device such as a computer or tablet, in proximity to a place where the individual sleeps; repeating the phrase directly prior to sleep to initiate the specific path; transcribing the outcome of the altered dream into the DAS plan; and comparing the gathered data of the recurring dream in need of alteration and the outcome of the altered dream with the DAS plan and with the DAS.

In a preferred embodiment, the DAS plan is a computer. In a more preferred embodiment, the key decision point in the recurring dream is elicited by a prompt on a computer. In a most preferred embodiment, the word or phrase that will alter the dream is determined by using a computer that provides suggestions.

In one embodiment, the dream is a nightmare.

In one embodiment, the recurring dream recurs over days to years.

In another embodiment, the phrase would consist of about two words.

In another embodiment, the phrase would consist of “let go,” “grab ladder,” “look within,” “be spontaneous,” “embrace color,” “turn on flashlight,” “new chapter,” “get ready,” “get packed,” “be intuitive,” “blow fog away,” “take ship,” “give in,” “stop struggling,” “surrender,” “think big,” “find balance,” “try left,” “maybe now,” “say it,” “dance,” or “don't say that.”

In another embodiment, the specific path is a path that is perceived negatively by the dreamer.

In yet another embodiment, the invention is directed to methods for altering negative emotions associated with recurring dreams comprising applying the method of the present invention while the negative emotions occur.

In another embodiment, the invention is directed to methods for improving productivity comprising applying the methods of the present invention to an individual in need to improved productivity.

In an embodiment, the invention relates to methods for altering a recurring dream comprising the steps of: identifying adults from a group of individuals wherein the adults are capable of making informed decisions; identifying a recurring dream in the adults for alteration wherein the recurring dream recurs for days to years; gathering data from the adults, wherein the data comprises a map of the dream in a timeline form; inputting the gathered data into the input unit of a computer; identifying an unresolved key decision point in the recurring dream by eliciting a response from the adults; identifying alternative paths from the unresolved key decision point by eliciting a response from the adults; choosing a specific path from among the alternative paths; inputting the selected path and phrase into the input unit of the computer; determining a phrase of about two words to initiate the specific path; creating an actionable dream strategy for modifying the outcome of the recurring dream, wherein the strategy includes the adults placing a physical copy of the plan, or a representation of the plan on a device such as a computer or tablet, in proximity to a place where the adults sleep; repeating the phrase directly prior to sleep to initiate the specific path; transcribing the outcome of the altered dream; and comparing the gathered data of the recurring dream in need of alteration and the outcome of the altered dream with the dream plan and with a central processing unit of the computer.

In another embodiment, key decision point is unresolved.

In yet another embodiment, the unresolved key decision point is a hanging scenario.

In a further embodiment, the unresolved key decision point is a struggling scenario.

The disclosed embodiments are simply exemplary embodiments of the inventive concepts disclosed herein and should not be considered as limiting, unless so stated.

### DESCRIPTION OF THE DRAWING

FIG. 1 is a representation of an embodiment of the method that comprises choosing a specific path from a KDP to allow dreams to continue freely once past the KDP.

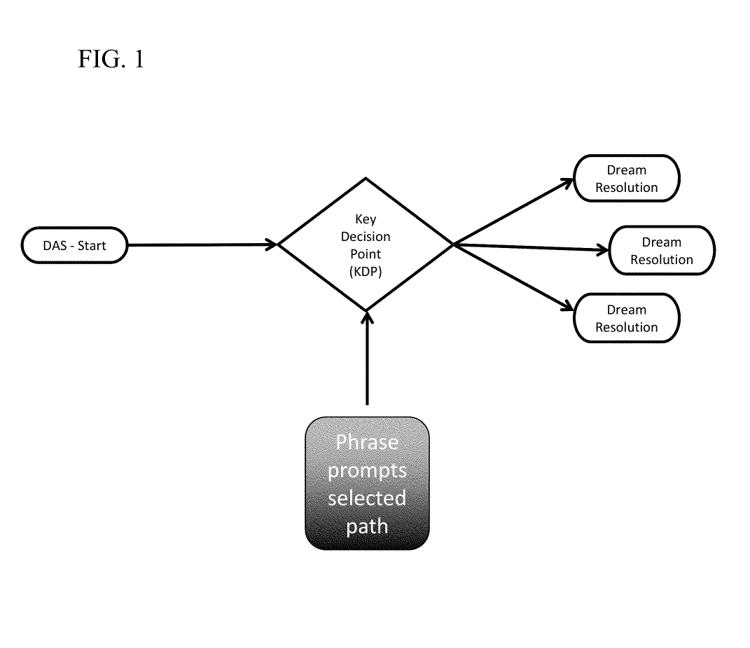

FIG. 2 is a representation of an embodiment of the method that comprises choosing a specific path from a KDP to allow dreams to alter or resolve.

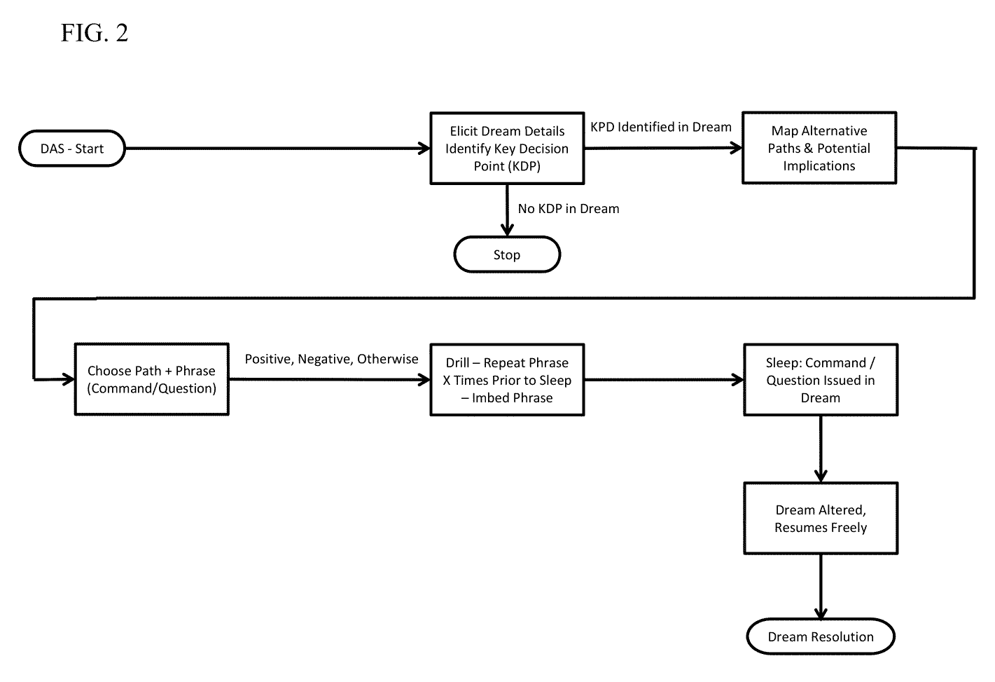

### DETAILED DESCRIPTION OF THE INVENTION

The present invention generally relates to a method of dream, including nightmare, management. Applicant has discovered that by using short phrases repeated directly prior to sleep a person can alter the outcome of dreams and even eliminate dreams altogether.

“Dreams” can be categorized but are not limited to:

(1) Non-Key Decision Point Dreams (Non-KDPDs) do not recur or do not contain a moment or juncture where a decision exists or may be prompted, or a key decision point. Non-KDPDs are likely part of the unconscious mind's way of keeping order and processing routine information and thoughts, general psychological maintenance such as attempting to resolve routine unresolved or imminent issues, reviewing or rehearsing situational scenarios, providing solutions to personal or even cultural challenges, sometimes inspiring innovation in the arts and sciences. Most Non-KDPDs either do not recur or recur only for short periods. Non-KDPDs range from seemingly real to abstract, goofy, and curious. Like all dreams, Non-KDPDs may or may not be remembered only in portions.

(2) Key Decision Point Dreams (KDPDs) serve many of the same purposes and tasks as Non-KDPDs, but within the dream is an identifiable moment or juncture where the dreamer is faced with a choice of some sort or a choice may be prompted, such as a literal or figurative fork in the road, whether to speak up in a meeting or not, etc. Hanging and struggling scenarios are included in this category. Some KDPDs recur for less than a week, while others may recur for the dreamer's lifetime.
(a) “Hanging scenario” refers to a scenario in which the KDP occurs while the dreamer is hanging from something such as a ledge, a railing, a cliff, an abstract object, etc. The KDP generally appears to end the dream as experienced to date. A hanging scenario KDPD is generally derived from intense feelings related to a series of events and interactions that created the feelings that created the dream (e.g., feelings of abandonment, such as from divorce or separation from a loved one) and not a specific identifiable event. The invention is particularly effective in this scenario because the KDP is not a fleeting moment; it is sustained for a period within the dream.
(b) “Struggling scenario” refers to a scenario in which the KDP occurs while the dreamer is in the midst of a struggle with a decision or a physical struggle with a person, animal, object, etc. A struggling scenario may or may not end a KDPD (e.g., PTSD derived dreams are considered struggling dreams and often do not end at the KDP when initially experienced, but may end or stick at the KDP when later experienced) and is caused by a specific unpleasant event combined with feelings associated with the event, the situation, the person, etc. The invention is particularly effective in this scenario if the KDP is not a fleeting moment and is sustained for a period within the dream.

“Alteration” or “altering” is defined as changing the course of or to lessen in severity or to eliminate.

The invention is directed to a method of altering recurring dreams, including nightmares. The invention can be utilized to “unstick” dreams that await resolution (resolution of a stuck dream is accomplished by unsticking it and allowing it to complete itself to the satisfaction of the dreamer), thus eliminating most recurring dreams and reducing the recurrence and severity of others. Specifically, the dreamer identifies a recurring dream and identifies a KDP in the recurring dream. The KDP is identified as a moment in the dream that the dreamer cannot move past or resolve or has difficulty doing so with satisfactory results. The dreamer then identifies a set of alternative paths the dream can take including paths the dreamer perceives as positive, negative or neutral. After choosing a specific path among the alternative paths, the dreamer determines a phrase that will initiate that specific path. Ideally, the phrase includes only a few words (e.g. “let go,” “grab ladder,” “look within,” “be spontaneous,” “embrace color,” “turn on flashlight,” “new chapter,” “get ready,” “get packed,” “be intuitive,” “blow fog away,” “take ship,” “give in,” “stop struggling,” “surrender,” “think big,” “find balance,” “try left,” “maybe now,” “say it,” “dance,” or “don't say that.”) The dreamer repeats this phrase as often as remembered. Ideally, the phrase is repeated soon before the dreamer goes to sleep to embed the phrase as a thought into the dreamer's unconscious in a priority position and with share of voice relative to other unconscious thoughts as the dreamer transitions to sleep.

The phrase, being embedded in the dreamer's unconscious thoughts, is then allowed to intersect with the recurring dream while both are playing in the dreamer's unconscious mind at night. The imbedding of the phrase into the unconscious thought and intersecting of the phrase with the recurring dream are accomplished without the need for external audio-to-conscious-to-unconscious translation and without regards to synchronizing the phrase in relation to the target dream by incorporating and bypassing the dreamer's indigenous defenses.

The invention uses short phrases with the specific intent of prompting the dream to move past the KDP towards the selected path and then allows the dream to continue freely, eliminating potential friction or interference with the dreamer's indigenous defenses inherent in longer scripted methods.

As used herein, the “Dream Alteration System plan” refers to a physical object that the dreamer may use to create their plan for altering their recurring dream's outcome. The plan may be a paper and pen, or a copy of the example plan below in Example 1 may be used to write down the dreamer's plan. A CD or DVD may have exercises or commands for the dreamer to listen to or watch to create their plan. A computer may also have the Dream Alteration System plan as a software program. This program could be interactive allowing the dreamer to create the plan my answering questions. The computer program may provide suggestions based on the dreamer's input. The plan may also be in a book by the author of this application which explains in more detail how to achieve successful resolution of the dreamer's dilemma by following the Dream Alteration System and creating a personal plan.

### Representative Embodiments

In a preferred embodiment, the present invention provides a method of altering a recurring dream, including a nightmare, comprising the steps of: identifying adults from a group of individuals wherein the adults are capable of making informed decisions; identifying a recurring dream in the adults for alteration wherein the recurring dream recurs for days to years; identifying an unresolved KDP in the recurring dream wherein the unresolved KDP is selected from the group of hanging scenario and struggling scenario; identifying alternative paths from the unresolved KDP wherein the alternative paths include both paths perceived by the dreamer as negative and paths perceived by the dreamer as positive; choosing a specific path from among the alternative paths; determining a phrase of about two words to initiate the specific path; and repeating the phrase directly prior to sleep to initiate the specific path.

### Example 1

A person has a recurring nightmare that seems to end while she is hanging from something such as a ledge or a cliff or the worst form: a large, dark, abstract cube over a deep dark endless abyss. In the dream state, the dreamer is watching herself, the heroine, from third party perspective as she struggles to hold on and survive; never failing by falling to her death or into a demon's clutches; never winning by climbing up and walking off into the sunset. For years after the nightmare was initially experienced, it caused the dreamer restless sleep, occasional sweaty mid-dream awakenings (night terrors), fear of sleeping and dreaming; and general anxiety or knots in her stomach. Over the years she has learned a great deal in her life; progressed with traditional therapies and overall is in a different place in life than when the nightmare first appeared. When she thinks about the nightmare, she believes that the feelings that led to the nightmare's creation are now truly residual. The nightmare occurs less frequently than it once did and it has not prevented her from leading a happy and fulfilled life but the severity largely remains when it does recur. Ultimately, at the end of the day the dreamer has difficulty understanding why the demons give her no quarter as they prey on her at night.

The invention of this application, otherwise known as the Dream Alteration System (“DAS”), calls for the person to identify the nightmare as a recurring dream that is in need of resolution. The person creates a map in timeline form of the dream by writing it down on paper. Next, the person identifies the KDP in the dream that may be used to allow the dream to resolve. In this case the KDP is what to do while hanging. After identifying the KDP, the person identifies the alternative paths available and any potential positive, negative or neutral implications of changing the dream. In this dream those alternative paths could include prompting for a ladder, a helicopter, a friend's hand or letting go and falling. From these alternative paths, the person must then choose the specific path that she wants to take. The person chooses to let go of the object intentionally, fall and take what comes. To make the choice the person then chooses a short phrase that could include “let go” or any short phrase that would determine the chosen path. The person in this scenario chooses “let go.” The person then repeats the phrase “let go” in her head as often as she can remember directly prior to sleep. The phrase “let go” becomes imbedded in her unconscious thoughts in a priority position with share of voice as the she drifts off to sleep. The thought “let go” then intersects with the recurring nightmare commanding the heroine to let go of the object. In the nightmare, the heroine opens her hands and starts to serenely fall and immediately the dream fades to black and ends. With any further recurrence of this dream, the dream will assume its new form without further repetition of the chosen phrase, with the heroine letting go at the appropriate moment in the dream; she is essentially vaccinated for life against this dream. The person will then enter the improved outcome of the dream into her DAS plan and compare the dream outcomes.

The chosen negative path was not ideal in terms of the outcome for the heroine within the dream movie; however, it leads to the resolution of the recurring nightmare by prompting the dreamer to move beyond the KDP. Given the intensely negative feelings that created this nightmare, falling may be the path her psyche originally intended for the heroine to take and is the path she may not have considered in prior attempts to resolve the dream. The recurring dream is eliminated by manipulating the mechanism of the dream itself that may then lead to further resolution of the feelings that caused the dream to occur by removing an intermittent reminder of those feelings and reducing doubt that they are sufficiently resolved. Taking control of the nightmare and changing it back into a harmless, almost silly dream dissipates general anxiety and reduces the knots she feels; anxiety and fear specifically related to the nightmare, sleeping, and dreaming are transformed to feelings of empowerment, control and freedom. Her dreams hold no horror for her anymore and she reflects this peace and calm in many subtle ways in her personal and professional lives.

A user of the system may use the plan below to facilitate her participation in the Dream Alteration System.

### Example Dream Alteration System Plan

Please reflect on each question below and record your answers below the questions in the provided space.

### Example 2

Through training and experience, a person has uncommonly vibrant dreams. He developed this skill so that he may use dreams in his profession for which he develops and writes stories. His various past projects intermittently continue to play as dreams, but mostly he experiences the dream of the project he is currently immersed in. The variability of the different versions of the dream tends to decrease as he fine-tunes the story to perfection. He is currently experiencing a recurring dream consistent with his current project. In the dream and in his story, the hero experiences the usual set up, catharsis and redemption, mostly while in outer space. The person's current project is going well overall, but his writer's gut is nagging at him. His audience may not feel the story entirely hangs together but he cannot quite put his finger on why or how to fix it.

The Dream Alteration System calls for the person to identify the dream as a recurring dream that is in need of resolution. The person then makes a map of the dream in timeline form and records it in his Dream Alteration System plan computer program. Next, the person identifies the KDP in the dream that needs to be made to allow the dream to resolve. In this case the KDP he identifies is a moment in the dream when the hero is about to exit the enemy vessel while his team is under attack. After identifying the KDP, the person identifies the alternative paths available and any potential positive, negative or neutral implications of changing the dream. In this dream those alternative paths could include materializing something, taking a different exit or continuing the fight. From these alternative paths, the person must then choose the specific path that he wants to take. Once this information is entered into the computer program, the computer program may make the suggestions to “drop and roll” or “look down”.

The person chooses to stop and look in a new direction in the dream and the resolution will be there. To make the choice the person chooses “drop and roll.” The person then repeats the phrase “drop and roll” in his head as often as he can remember directly prior to sleep. The phrase “drop and roll” becomes imbedded in his unconscious thoughts in a priority position. The thought “drop and roll” then intersects with the recurring dream commanding the hero to drop and roll smartly in the dream, ultimately putting the hero in a slightly different place than before and looking left for the first time. When the hero looks left in his dream, his psyche then inserts the following: a demure “enemy” princess has been shadowing our hero. She reveals herself holding a scarf and a scroll with plans for a top-secret peace weapon. The hero kisses the princess, snatches the scroll as the princess ties the scarf around his neck, saves the universe, saves the writer's story and lives for the sequel. The writer transcribes the outcome into the DAS plan computer program. He then compares the outcomes of the dreams.

The writer likes the twist, develops this path further using his dreams and finishes the story to his satisfaction.

Resolution of the recurring dream occurred by prompting the dreamer to move beyond the KDP in a direction that was different than previously experienced and to the satisfaction of the dreamer. This person may feel professional pride and contentment and continue to use his dreams productively.

### Example 3

A person has a recurring nightmare that as he is hiking up a hill he trips over a rock, which in turn loosens a rather large boulder. In the dream state, the person grabs the boulder to stop it from rolling down the hill and smashing his truck. The person struggles with the boulder because he is not strong enough to move it to a secure place but does not want to let go of it. In the conscious state, the person has been meeting often with his business partner about expanding their company and is hesitant about moving forward with a specific plan. The business partner has proposed plans include buying out a competitor's company or merging with that company and dissolving their current company. He feels reluctant to let go of the company he spent so much time building but is also averse to destroying another person's company.

The Dream Alteration System calls for the person to identify the nightmare as a recurring dream that is in need of resolution. The person creates a map of the dream in timeline form using his Dream Alteration System plan book or DVD. Next, the person identifies the KDP in the dream that needs to be made to allow the dream to resolve. In this case, the KDP is what to do with the boulder. After identifying the KDP, the person identifies the alternative paths available and any potential positive, negative or neutral implications of changing the dream. In this dream those alternative paths could include allowing the boulder to smash his truck or diverting the boulder just enough to allow it to roll down another face of the hill and smash another hiker's truck. From these alternative paths, the person must then choose the specific path that he wants to take. The person chooses to allow the boulder to smash his own truck. To make the choice the person reads the book or watches the DVD for suggestions that could include “let go,” “stop struggling,” “move over,” or any short phrase that would determine the chosen path. The person in this scenario chooses “let go.” The person then repeats the phrase “let go” either in his head or out loud as often as he can remember directly prior to sleep. The phrase “let go” becomes imbedded in his unconscious thoughts in a priority position. The thought “let go” then intersects with the recurring nightmare commanding the person to let go of the boulder and in turn, the boulder smashes his truck. The alternate phrase “move over” may result in equal net resolution by allowing the person to move beyond the KDP, however the two altered dreams may range from identical to largely different from one another in terms of their specific content; for example whether the boulder rolls over the dreamer on its path to the truck or whether it does not. The person then records the outcome of the dreams and compares them.

The chosen negative path was not ideal from the truck's perspective or the dreamer's emotions related to his truck's destruction but it leads to the resolution of the recurring nightmare by prompting the dreamer to move beyond the KDP. The dreamer may have an intense feeling of relief that he allowed the boulder to smash his truck rather than diverting it towards the other hiker's truck. The person carries over this feeling to his meetings with his business partner where he makes the major decision of merging with the competitor company. The recurring dream was eliminated by treating the dream itself, which then led to resolution of the feelings and situation that likely caused the dream to occur.

The disclosed embodiments and examples are simply exemplary embodiments and examples of the inventive concepts disclosed herein and should not be considered as limiting, unless so stated.

What is claimed is:

1. A method of altering a recurring dream comprising the steps of:
i. identifying an individual that has a recurring dream in need of alteration;
ii. gathering data from the individual, wherein the data comprises a map of the dream in a timeline form;
iii. inputting the gathered data into a Dream Alteration System (“DAS”) plan, wherein the DAS plan is presented in a form selected from the group consisting of a computer, book, CD, patent, patent application publication, and DVD;
iv. identifying a key decision point in the recurring dream by eliciting a response from the individual and referencing the DAS for suggestions;
v. identifying alternative paths from the key decision point by eliciting a response from the individual;
vi. choosing a specific path from among the alternative paths;
vii. determining a phrase to initiate the specific path by referencing the DAS plan for suggestions and eliciting a response from the individual;
viii. inputting the selected path and word or phrase into the DAS plan;
ix. creating an actionable dream strategy for modifying the outcome of the recurring dream, wherein the strategy includes the individual placing a physical copy of the DAS plan, or a representation of the plan on a device such as a computer or tablet, in proximity to a place where the individual sleeps;
x. repeating the selected word or phrase directly prior to sleep to initiate the specific path;
xi. transcribing the outcome of the altered dream into the DAS plan; and
xii. comparing the gathered data of the recurring dream in need of alteration and the outcome of the altered dream with the DAS plan and with the DAS.

2. The method of claim 1, wherein the DAS plan is a computer.

3. The method of claim 2, wherein the key decision point in the recurring dream is elicited by a prompt on a computer.

4. The method of claim 2, wherein the short phrase that will alter the dream is determined by using a computer that provides suggestions.

5. The method of claim 1, wherein the dream is a nightmare.

6. The method of claim 1, wherein the recurring dream recurs over days to years.

7. The method of claim 1, wherein the phrase consists of about two words.

8. The method of claim 7, wherein the phrase is selected from the group consisting of “let go,” “grab ladder,” “look within,” “be spontaneous,” “embrace color,” “turn on flashlight,” “new chapter,” “get ready,” “get packed,” “be intuitive,” “blow fog away,” “take ship,” “give in,” “stop struggling,” “surrender,” “think big,” “find balance,” “try left,” “maybe now,” “say it,” “dance,” and “don't say that.”

9. The method of claim 1, wherein the key decision point is unresolved.

10. The method of claim 9, wherein the unresolved key decision point is a hanging scenario.

11. The method of claim 9, wherein the unresolved key decision point occurs in a struggling scenario.

12. The method of claim 1, wherein the specific path is a path that is perceived negatively by the dreamer.

13. A method of altering negative emotions associated with recurring dreams comprising applying the method of claim 1 while the negative emotions occur.

14. A method of improving productivity comprising applying the method of claim 1.

15. A method of altering a recurring dream comprising the steps of:
i. identifying adults from a group of individuals wherein the adults are capable of making informed decisions;
ii. identifying a recurring dream in the adults for alteration wherein the recurring dream recurs for days to years;
iii. gathering data from the adults, wherein the data comprises a map of the dream in a timeline form;
iv. inputting the gathered data into the input unit of a computer;
v. identifying an unresolved key decision point in the recurring dream by eliciting a response from the adults;
vi. identifying alternative paths from the unresolved key decision point by eliciting a response from the adults;
vii. choosing a specific path from among the alternative paths;
viii. inputting the selected path and phrase into the input unit of the computer;
ix. determining a phrase of about two words to initiate the specific path;
x. creating an actionable dream strategy for modifying the outcome of the recurring dream, wherein the strategy includes the adults placing a physical copy of the plan, or a representation of the plan on a device such as a computer or tablet, in proximity to a place where the adults sleep;
xi. repeating the phrase directly prior to sleep to initiate the specific path;
xii. transcribing the outcome of the altered dream; and
xiii. comparing the gathered data of the recurring dream in need of alteration and the outcome of the altered dream with the dream plan and with a central processing unit of the computer.

16. The method of claim 15, wherein the unresolved key decision point is a hanging scenario.

17. The method of claim 15, wherein the unresolved key decision point is a struggling scenario.

18. The method of claim 15, wherein the specific path is a path that is perceived negatively by the dreamer.
